(19) United States
(12) Patent Application Publication
(10) Pub. No.: US 2005/0209568 A1
(43) Pub. Date: Sep. 22, 2005

(54) Needle cap assembly for syringe

(76) Inventor: Laurence M. Shanley, Miami, FL (US)

Correspondence Address: LERNER AND GREENBERG, PA, P O BOX 2480, HOLLYWOOD, FL, 33022-2480, US

(21) Appl. No.: 10/802,354

(22) Filed: Mar. 16, 2004

(51) Int. Cl. A61M 5/32

(52) U.S. Cl. 604/198

(57) ABSTRACT

A safety needle cap for a syringe completely covers the hypodermic needle during non-use or between injections. The needle can be easily exposed with a reduced danger of accidental needle-stick injury. The needle cap can be unlocked from the closed position by being rotated, and then slid backwards onto the syringe barrel by a defined travel distance. Several settings, up to the full extension of the needle, are possible. After use, the needle retracted into the cap, once more preventing needle-stick injury.

### BACKGROUND OF THE INVENTION

1. Field of the Invention

The invention relates to medical sharps devices. More specifically the invention relates to a protective device for a hypodermic needle and syringe.

2. Description of the Related Art

Accidental sharps contamination, such as needle-stick injuries from syringes are all too common. The contamination and infection dangers associated with such accidents are commonly feared in the medical field. Considerable concerns are associated with the potential for HIV and hepatitis infection, to name just two.

A variety of sharps protectors are described in the art. For instance, U.S. Pat. No. 6,699,223 B2 to Sharp describes a safety syringe in which the needle assembly is stored in the syringe barrel after use. U.S. Pat. No. 6,692,470 B2 to Sanpietro describes a syringe assembly with a cylindrical shield covering the needle. For the purpose of using the syringe, the cylindrical shield is pulled back onto the barrel, thus exposing the needle. U.S. Pat. No. 6,616,638 B2 to Peters, III, describes a hypodermic needle cap. Prior to using the syringe, the cap is popped off to expose the needle.

While the prior art systems provide a certain amount of safety against sharps accidents, further improvements are nevertheless desirable.

### SUMMARY OF THE INVENTION

It is accordingly an object of the invention to provide a protective cap for a sharps device, in particular a needle cap and hypodermic needle assembly for a syringe, which overcomes the above-mentioned disadvantages of the heretofore-known devices and methods of this general type and further improve the device with regard to the dangers of needle-stick and similar sharps injuries.

With the foregoing and other objects in view there is provided, in accordance with a general concept of the invention, a protective cap assembly for a sharps device. The assembly comprises:
a receiver for rigidly holding a sharps element of the sharps device;
a protective cap assembly attached to the receiver and completely encasing the sharps element in a closed position of the cap assembly;
the receiver being movably disposed in the protective cap assembly, for movement from the closed position to a functional position in which the sharps element projects from the protective cap assembly and the sharps device is in a functional condition, and from the functional position to the closed position in which the sharps element is completely retracted in the protective cap assembly.

In accordance with an added feature of the invention, the protective cap assembly includes a clip ring and a protective cap attached to the clip ring, and wherein the clip ring is configured to limit a movement of the receiver in one direction and the cap is configured to limit the movement of the receiver in another direction.

In accordance with a preferred embodiment of the invention, the receiver has a tab formed on a substantially cylindrical jacket surface thereof, and the protective cap assembly is formed with at least one groove in an inner jacket surface thereof, defining a track within which the tab slides from the locked position to the functional position.

In the preferred embodiment of the invention, sharps device is a syringe and the sharps element is a hypodermic needle.

With the above and other objects in view there is also provided, in accordance with the invention, a needle cap assembly for a syringe having a distal end and a hypodermic needle projecting from the distal end. The needle cap assembly comprises:
a receiver rigidly mountable at the distal end and rigidly holding the hypodermic needle;
a protective cap mounted on the receiver and slidable relative to the receiver between a closed position in which the protective cap encases the hypodermic needle completely and a functional position in which the hypodermic needle projects out of the protective cap; and
mutually cooperating locking devices on the protective cap and on the receiver for locking the protective cap in the closed position.

In accordance with an additional feature of the invention, the protective cap has a tip formed with an opening through which the needle projects in the functional position, and a membrane covering and substantially sealing the opening when the protective cap is in the closed position and the needle is completely retracted inside the cap.

With the above and other objects in view there is further provided, in accordance with the invention, a syringe assembly, comprising a syringe having a plunger and a barrel with a distal end, the needle cap assembly as described in the foregoing, and a needle held in the receiver of the needle cap assembly. The needle is mounted, together with the receiver and the needle cap assembly, to the distal end of the barrel.

In accordance with a concomitant feature of the invention, syringe has a standard luer lock and the needle cap assembly and the needle together are formed to be mounted on the luer lock.

The safety needle cap and needle assembly provides for a simple and safe means to prevent needle-stick injuries, particularly in the context of intramuscular injections. The device is particularly advantageous in that it is compatible with common preexisting syringe needle combinations. The product includes safety features that leave very little room for error in the high-stress, fast-paced setting typically found in medical treatment facilities.

The novel device is configured to accommodate all regular syringe needle functions, including safe disposal. The assembly is at the same time a needle cap—for needle coverage prior to use and after use—and permits full function of the syringe in that the needle may be selectively exposed at a variety of positions. That is, the device permits withdrawing medication, transportation, storage, injection, and even re-use for multiple injections. The entire procedure is possible without even exposing the needle to open sight.

The device is particularly suited for needle gauges in the range from 19-25. Other sizes, of course, are possible as well. In a preferred embodiment, the device is configured for use with standard 1, 3, and 5 cc syringes. As will be seen from the following description, the length of the needle is of little import and the device is usable with full-length needles, yet can be utilized with several settings that provide for the functionality of different length needles.

In a preferred embodiment, the assembly can be packaged as a unit with the needle inside the cap, and with or without the syringe.

Other features which are considered as characteristic for the invention are set forth in the appended claims.

Although the invention is illustrated and described herein as embodied in a needle cap for a syringe, it is nevertheless not intended to be limited to the details shown, since various modifications and structural changes may be made therein without departing from the spirit of the invention and within the scope and range of equivalents of the claims.

The construction of the invention, however, together with additional objects and advantages thereof will be best understood from the following description of the specific embodiment when read in connection with the accompanying drawings.

### BRIEF DESCRIPTION OF THE DRAWINGS

FIG. 1 is a perspective view of the needle cap assembly according to the invention in combination with a syringe shown in a closed position;

FIG. 2 is a perspective view of the needle cap assembly and the syringe shown in a half position;

FIG. 3 is a perspective view of the needle cap assembly and the syringe shown with the needle in a fully extended position;

FIG. 4 is a top perspective, exploded view of the entire assembly according to the invention;

FIG. 5 is a bottom perspective, exploded view of the needle cap safety assembly;

FIG. 6 is a sectional view through the clip ring and a detail excerpt of its wall with a locking tab;

FIG. 7 is a sectional view of the receiver of the assembly, without the hypodermic needle;

FIG. 8 is a sectional view of needle cap of the assembly; and

FIG. 9 is an exploded X-ray view of the complete assembly according to the invention, including the syringe.

### DESCRIPTION OF THE PREFERRED EMBODIMENTS

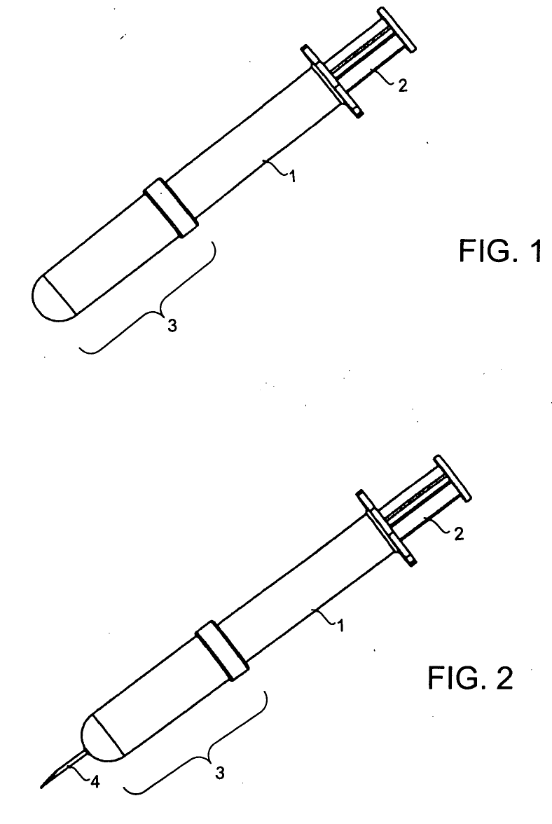

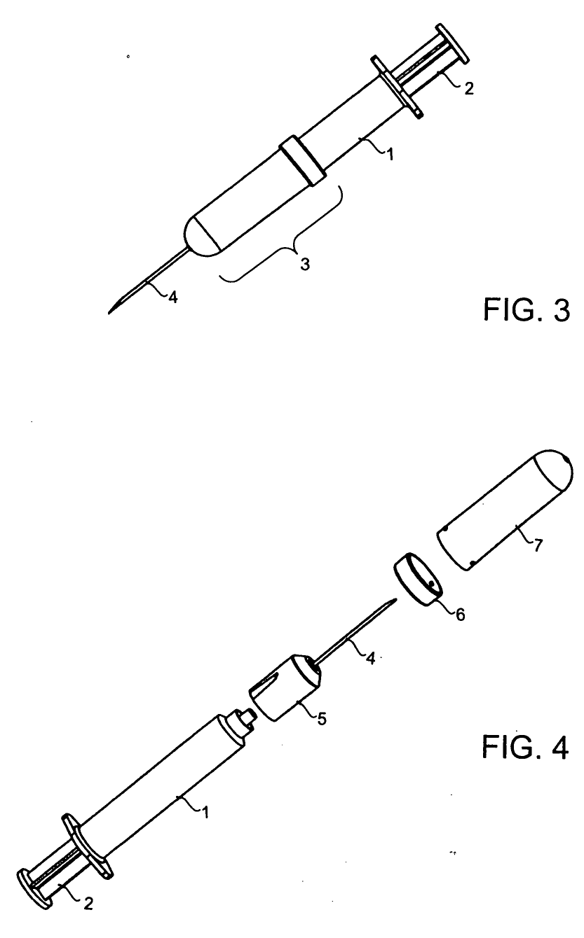

Referring now to the figures of the drawing in detail and first, particularly, to FIGS. 1-3 thereof, there is seen a syringe with a barrel 1 and a piston 2. The syringe is a standard device with, say, a 3 cc barrel. The syringe to be used with the invention is typically in the range from 1 to 5 cc. A distal end of the barrel 1, with reference to the plunger handle, carries a needle cap safety assembly according to the invention. While the needle 4 does not show in FIG. 1—i.e., it is completely retracted—the needle 4 is half-way exposed in FIG. 2 and it is fully extended in FIG. 3. As will be seen from the following, the relative projection of the needle 4 from the needle safety cap is achieved by sliding the needle cap back onto the barrel 1.

With reference to FIG. 4, the needle cap safety assembly 3 comprises a receiver or needle holder 5 with a standard luer lock, the needle 4, a ring clip 6, and a sleeve cap 7.

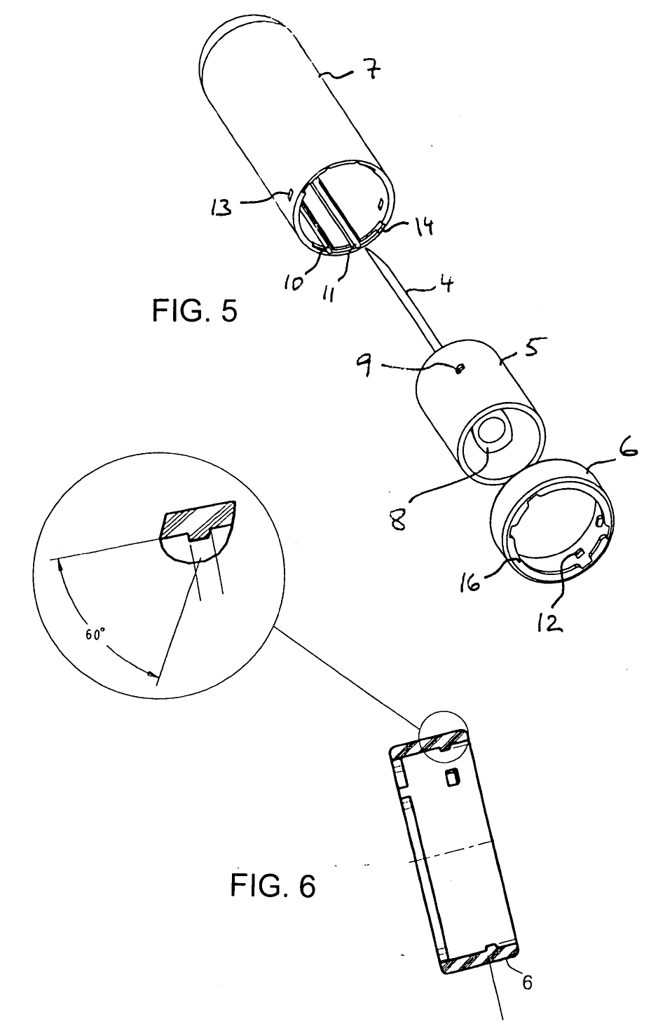

With reference to FIG. 5, a luer lock 8 is integrated in the receiver 5, so that the receiver 5 with the needle 4 can be connected to a standard luer lock at the distal end of the syringe barrel 1. Two tabs 9 project from the cylindrical jacket surface of the receiver 5, at diametrically opposite points. The tabs 9 are configured to slide in tracks 10 and 11 formed in the inner wall surface of the cap 7 when the receiver is inserted. The outer diameter of the receiver 5 is adapted to the inner diameter of the cap 7 so as to assure good slidability, yet an otherwise rigid fit to provide for a relatively stable connection. In a preferred embodiment of the invention, the outer diameter of the receiver is 0.540″ and the inner diameter of the cap 7 is 0.555″ in one direction and 0.580 in a perpendicular direction. That is, one of the two jackets (inner surface of the cap 7 or outer jacket surface of the receiver 5) may be formed slightly unround or elliptical. This aids in the functionality of the devices and provides for a better functional lock between the two components.

The ring clip 6 is also formed with several tabs 12. The tabs 12 project from the inner wall surface and are configured to lock into corresponding openings 13 formed in the cap 7. The tabs 12 and the openings 13 (through holes or blind bores) are formed so that the easily snap into one another during the assembly of the device, yet do not easily come apart during use. In a preferred embodiment, as illustrated in FIG. 6, the tab 12 is formed with a draft angle of approximately 30° to enable insertion and snapping into the opening 13. The backside of the tab 12, i.e., the side facing the plunger handle of the syringe, is formed with a 90° angle. This ensures proper locking of the snap ring on the cap 7.

In the alternative, or in addition, any of several attachment processes are possible. For example, the ring clip 6 may be glued to the cap 7 or welded by common vulcanization or with ultrasonic welding. Any or all of these options may be chosen, in any combination.

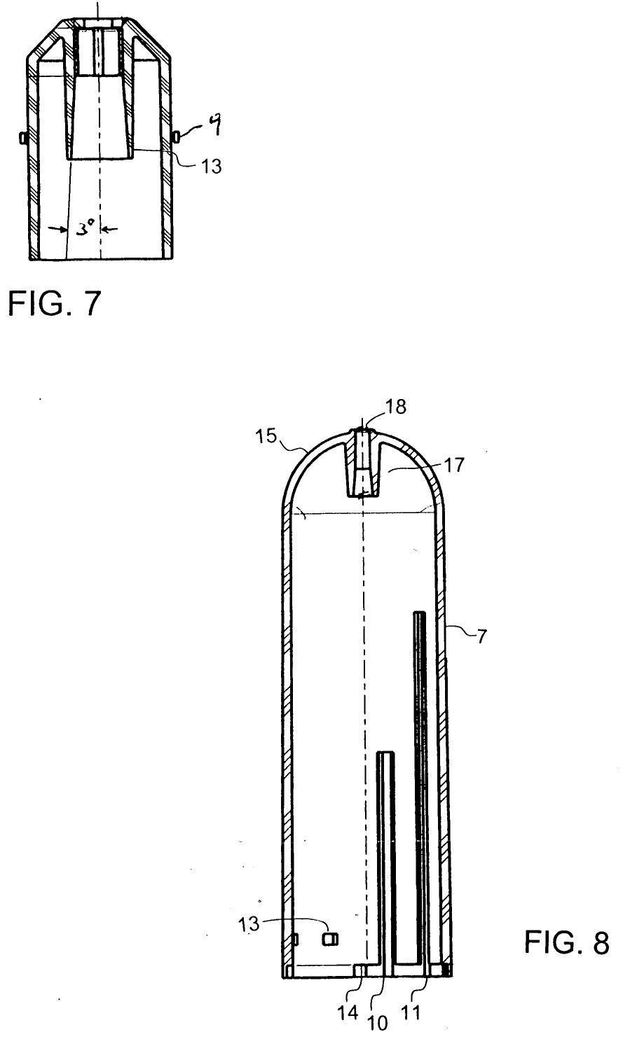

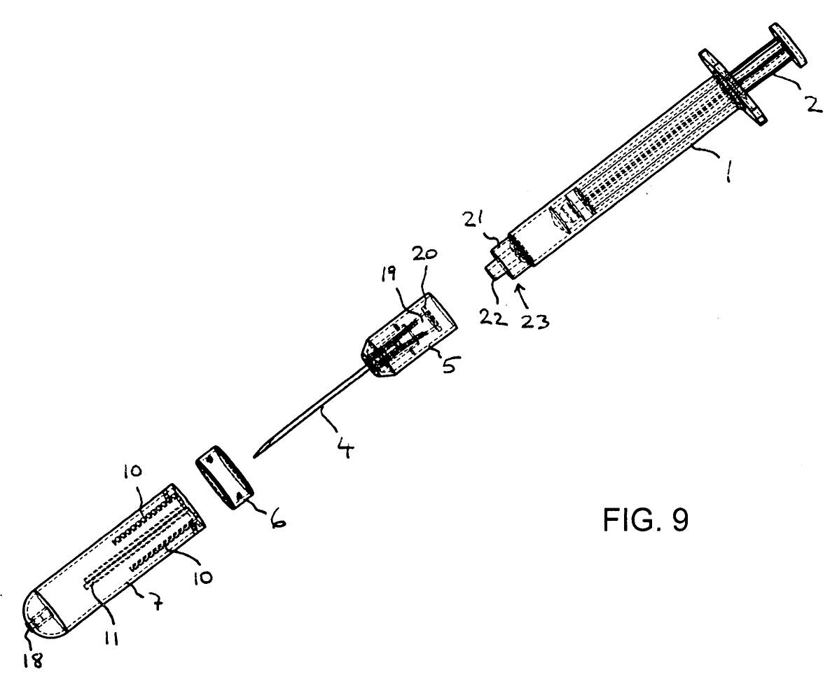

With reference to FIG. 7, the receiver 7 has a conical stub 13 into which a base of a needle is inserted and locked. The conicity of the inner surface of the stub 13 may be defined by a draft angle of approximately 3°, thus assuring proper hold for the needle base. The outside of the needle base is conventionally formed with a thread ridge or a tread tab so as to enable it to be threaded into the standard luer lock tread at the distal end of the syringe. The receiver and the needle base, as well as the needle itself, may be integrally formed, with the needle directly integrated into the mold of the receiver. Alternatively, the needle may be inserted and glued into the receiver after its release from the mold.

With reference to FIG. 8, the cap 7 is formed with the two above-mentioned sliding tracks 10 and 11, as well as a locking track 14. The locking track 14 sets the full extension of the cap 7 and thus hides the needle 4 inside the cap 7. The track 10 allows half-way extension, i.e., a “short” needle will emerge from the tip 15 of the cap 7. The track 11 allows the cap 7 to be slid back onto the barrel to is full extent, i.e., a “long” needle will emerge from the tip 15. The short needle setting may be used, for example, for drawing medication, for shallow muscle injections or for petite persons. The long needle setting would typically be used for deep muscular injection.

The receiver 5 is prevented from sliding backwards, out of the cap 7 beyond its locked position in the track 14 by a backwall 16 of the clip ring 6. As the receiver 5 slides forward inside the cap 7, the needle tip emerges from the tip 15. A needle guide 17 is provided for that purpose. The needle guide 17 has a conical entry segment followed by a cylindrical stabilization segment. The latter has a diameter corresponding to the largest rated needle diameter. The top of the cap is formed with a membrane 18, which may be a simple plastic foil or a spun-on plastic that ruptures as the needle emerges from the tip. The membrane 18 assures that the needle is completely encased and protected from contamination during shipping and storage prior to use. If the device is used for multiple injections, as the needle is retracted, the membrane closes and once more protects the needle against contamination during the interim periods.

With reference to FIG. 8, there is illustrated an exploded X-ray view of the complete assembly, including the syringe. As shown, the needle base 19 has a thread ridge 20 the meshes with an inner thread 21 at the distal end of the syringe. The luer lock stem 22 is configured to lock into the base 19. The distal end of the syringe with the luer lock, generally identified with numeral 23, slides into the receiver 5. As shown inside the cap 7, the tracks 10 and 11 are multiplied.

It is possible to use the new syringe and needle cap assembly without openly exposing the needle 4. For example, if medication is to be withdrawn from a container to fill the barrel, the tip 15 is placed onto the container, with the membrane 18 aligned with the membrane of the medication container. The cap 7 is then rotated so as to move the tab or tabs 9 from the locking track 14 into alignment with the sliding track 10 or 11. Since only a short needle is necessary for the withdrawal operation, the alignment with the track 10 is sufficient. The tip 15 remains in contact with the top of the container. After filling the barrel to the desired level, the cap 7 is pushed forward while the syringe and the needle are being retracted. As the needle pulls out of the container, it also retracts beyond the membrane 18 and into the cap 7. Similarly, if an injection is be administered, the tip 5 of the cap 7 is placed onto the skin, the needle is then driven out to the desired length—and at the same time driven into the patient's tissue—all the while with the tip remaining in contact with the skin. During the withdrawal of the needle, once more, the tip 15 remains in contact with the skin, until the needle is hidden inside the cap 7.

The various rotational positions may be adapted to the specific use of the device. For instance, in the preferred embodiment, the locking position and the sliding positions (tracks 10, 11) are set so that, when the cap protector assembly is unscrewed from the syringe, the assembly is automatically set to the locking position with the needle completely retracted inside the inner space. This provides for a sharps protector with added safety.

Also, the inside of the cap 7 is formed with a draft angle such that, when the receiver 5 is fully pushed forward (the needle 4 is completely extended), the receiver 5 is friction locked in the cap 7. This makes for a very stable assembly during its use.

The advantages of the novel device are immediately apparent. By way of example, the device provides for safe medication withdrawal with a clearly reduced likelihood of a needle-stick injury. Safe and sterile medication storage is assured until the timing of the actual injection and between partial injections, because the safety cap completely encloses the sharps element. The full enclosure also assures safe transportation to the patient or laboratory. The injection can be effected without ever exposing the needle to the patient. The device can replicate two or more needle sizes for a variety of injection depths. Finally, the device can be locked and thus protected against accidental sharps injury following the injection.

The novel device is suitable for a variety of applications. For instance, all intramuscular injections can be performed, as well as all I.V. injections, and lidocaine injections. Finally, as noted above, the device is well suited for medication withdrawal.

1. A protective cap assembly for a sharps device, comprising:
a receiver for rigidly holding a sharps element of the sharps device;
a protective cap assembly attached to said receiver and completely encasing the sharps element in a closed position of the cap assembly;
said receiver being movably disposed in said protective cap assembly, for movement from the closed position to a functional position in which the sharps element projects from said protective cap assembly and the sharps device is in a functional condition, and from the functional position to the closed position in which the sharps element is completely retracted in said protective cap assembly.

2. The assembly according to claim 1, wherein said protective cap assembly includes a clip ring and a protective cap attached to said clip ring, and wherein said clip ring is configured to limit a movement of said receiver in one direction and said cap is configured to limit the movement of said receiver in another direction.

3. The assembly according to claim 2, wherein said receiver has a tab formed on a substantially cylindrical jacket surface thereof, and said protective cap assembly is formed with at least one groove in an inner jacket surface thereof, defining a track within which said tab slides from the locked position to the functional position.

4. The assembly according to claim 1, wherein the sharps device is a syringe and the sharps element is a hypodermic needle.

5. A needle cap assembly for a syringe having a distal end and a hypodermic needle projecting from the distal end, the needle cap assembly comprising:
a receiver rigidly mountable at the distal end and rigidly holding the hypodermic needle;
a protective cap mounted on said receiver and slidable relative to said receiver between a closed position in which the protective cap encases the hypodermic needle completely and a functional position in which the hypodermic needle projects out of said protective cap; and
mutually cooperating locking devices on said protective cap and on said receiver for locking said protective cap in the closed position.

6. The needle cap assembly according to claim 5, wherein said protective cap has a tip formed with an opening through which the needle projects in the functional position, and a membrane covering and substantially sealing said opening when the protective cap is in the closed position and the needle is completely retracted inside said cap.

7. The needle cap assembly according to claim 5, which comprises a clip ring connected with said protective cap, and wherein said clip ring is configured to limit a movement of said receiver in one direction and said protective cap is configured to limit the movement of said receiver in another direction.

8. The needle cap assembly according to claim 7, wherein said receiver has a tab formed on a substantially cylindrical jacket surface thereof, and said protective cap is formed with at least one groove in an inner jacket surface thereof, defining a track within which said tab slides from the locked position to the functional position.

9. A syringe assembly, comprising a syringe having a plunger and a barrel with a distal end, the needle cap assembly according to claim 5, a needle held in said receiver and mounted, together with said receiver and said needle cap assembly, to said distal end of said barrel.

10. The syringe assembly according to claim 9, wherein said distal end of said barrel is formed with a luer lock and said needle cap assembly and said needle together are formed to be mounted on said luer lock.
